(19) United States
(12) Patent Application Publication
(10) Pub. No.: US 2023/0414723 A1
(43) Pub. Date: Dec. 28, 2023

(54) ENHANCED HYT-INDUCED PROTEIN DEGRADATION USING LIPID NANOPARTICLE DELIVERY

(71) Applicant: Trustees of Tufts College, Medford, MA (US)

(72) Inventors: Qiaobing Xu, Medford, MA (US); Jinjin Chen, Medford, MA (US)

(21) Appl. No.: 18/033,492

(22) PCT Filed: Oct. 26, 2021
(86) PCT No.: PCT/US21/56635

§ 371 Date: Apr. 24, 2023

Related U.S. Application Data
(60) Provisional application No. 63/105,610, filed on Oct. 26, 2020.

(51) Int. Cl. A61K 38/45; A61K 31/5517; A61K 31/426; A61P 35/00

(52) U.S. Cl. CPC A61K 38/45; A61K 31/5517; A61K 31/426; A61P 35/00; C12Y 203/02

(57) ABSTRACT

Disclosed are conjugates, comprising a PROTAC and a protein. Also disclosed are nanoparticles, comprising the conjugates disclosed herein and a membrane. The present disclosure further relates to therapeutic methods of using the conjugates and nanoparticles.

### RELATED APPLICATIONS

This application claims the benefit of priority to U.S. Provisional Patent Application No. 63/105,610, filed on Oct. 26, 2020.

### GOVERNMENT SUPPORT

This invention was made with government support under grant number EB027170-01 awarded by the National Institutes of Health. The government has certain rights in the invention.

### BACKGROUND

Targeted degradation of specific proteins via the ubiquitin-proteasome system (UPS) by small molecules proteolysis-targeting chimaera (PROTAC) has emerged as a promising strategy for treating various diseases, owing to their high efficiency and selectivity. PROTACs are hetero bifunctional molecules composed of two components, a ligand targeting a protein of interest (POI) and an UPS activating ligand, connected by a linker. ARV-110, an orally administrated PROTAC for androgen receptor (AR) degradation, is currently in clinic trials for the treatment of prostate cancer, demonstrating the great clinical potential of technologies based on the UPS-mediated degradation of POI. However, even though PROTACs show significant advantages compared with small molecule inhibitors, there are two obstacles hindering their further development.

First, the cell permeability of the PROTAC is decreased because of increased molecular weight (MW>800) and the presence of multiple hydrogen bond donors and acceptors. Furthermore, the degradation of POIs by PROTACs requires the formation of an active ternary complex, which is a three-component system. As a result, the degradation ability of PROTAC decreased dramatically when applied at a high concentration due to the ‘hook effect’ of the three-component system. However, there are few studies discussing the methods of overcoming these drawbacks.

Moreover, apart from the lowing cell permeability, the “hook effect” remains a drawback in its own right. In the three-component system, the successful formation of activated ternary complex by PROTACs requires the conjugate of both a POI and an E3-ligases related protein (E3Ps).

In view of the foregoing, there is an unmet need for new PROTACs and compositions related thereto.

### SUMMARY OF THE INVENTION

In one aspect, the present disclosure provides conjugates comprising a proteolysis-targeting chimera (PROTAC) and a first protein, wherein the PROTAC and the first protein are covalently or non-covalently bonded to each other; and the first protein initiates degradation of a second protein.

In another aspect, the present disclosure provides nanoparticles, comprising a conjugate disclosed herein, and a membrane encapsulating the conjugate.

In another aspect, the present disclosure provides pharmaceutical compositions, comprising a conjugate or nanoparticle disclosed herein, and a pharmaceutically acceptable excipient.

In another aspect, the present disclosure provides methods of treating diseases or disorders, comprising administering to a subject in need thereof a therapeutically effective amount of a conjugate or nanoparticle disclosed herein.

### BRIEF DESCRIPTION OF THE DRAWINGS

FIG. 1A is a scheme for delivery of pre-fused PROTACs. The formation of active ternary complex of two or three component system calculated by theoretical models.

FIG. 1B is a scheme for delivery of pre-fused PROTACs. The LNP delivers the pre-fused PROTAC for enhanced activation of the ubiquitin-proteasome system through one-step binding instead of two step binding with free PROTAC.

FIG. 2A is a TEM image of a 80-O14B LNP complex.

FIG. 2B is a TEM image of 80-O14B/SARD279-HSP70 LNP complex.

FIG. 3A is a Western blot showing AR expression under different formulations of LNP, SARD279, and HSP70.

FIG. 3B is a Western blot showing concentration depended AR degradation by pre-fused SARD279.

FIG. 3C shows the structure of pre-fused SARD279 with 80-O14B, a lipid membrane.

FIG. 4A is a Western blot showing BRD4 expression in Hela cells by free ARV-825 at different concentrations.

FIG. 4B is a Western blot showing BRD4 expression in Hela cells after treatment of different formulations.

FIG. 4C shows the structure of pre-fused ARV-771 with 80-O14B, a lipid membrane.

FIG. 5A is a Western blot of the time-dependent degradation of BRD4 by free ARV-771 after incubation for a different period of time.

FIG. 5B is a Western blot of the time-dependent degradation of BRD4 by pre-fused ARV-771 after incubation for a different period of time.

FIG. 6 shows the chemical structure of 80-O14B

### DETAILED DESCRIPTION OF THE INVENTION

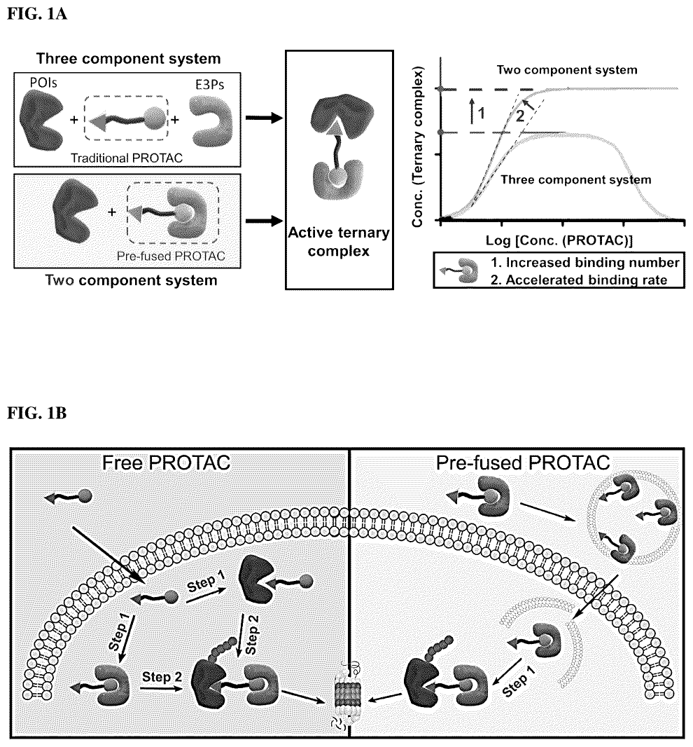

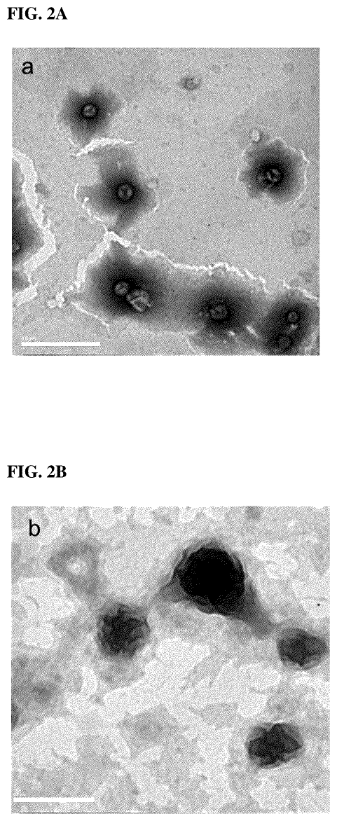

In a theoretical model, a two component system shows higher binding amount and rate constant as compared to a three-component system (FIG. 1A). Therefore, if the three-component PROTAC system can be transformed to a two-component system, the degradation efficiency of POIs is predicted to be significantly increased.

Compared with using PROTAC alone, there are several advantages to using nanoparticles to deliver PROTAC for protein degradation, such as i) enhanced intracellular delivery, ii) increased organ- and cell-specific targeting and accumulation, iii) synergistic effect of the nanoparticle and PROTAC in protein degradation, iv) encapsulating multiple agents into same nanoparticles, v) improve the bioavailability and formulation challenge for some PROTAC molecules, and vi) improve protein degradation efficiency by delivering small molecule/ubiquitin-related protein conjugates.

In one aspect, disclosed herein is a lipid nanoparticle (LNP) platform used to deliver the incorporated E3Ps and PROTACs (termed pre-fused PROTACs) which improves the degradation of POI as compared to those treated with PROTACs only. The pre-fused PROTAC forms ternary complex more effectively than a traditional PROTAC. The pre-fused PROTAC was then encapsulated into LNP and which possess several advantages, such as evading the endo/lysosome after the endocytosis (FIG. 1B).

In one aspect, the present disclosure provides conjugates comprising a proteolysis-targeting chimera (PROTAC) and a first protein, wherein the PROTAC and the first protein are covalently or non-covalently bonded to each other; and the first protein initiates degradation of a second protein.

In certain embodiments, PROTAC and the first protein are covalently bonded to each other. In certain embodiments, the PROTAC and the first protein are non-covalently bonded to each other.

In certain embodiments, the first protein initiates the degradation of the second protein via the ubiquitin-proteasome system (UPS). In certain embodiments, the first protein initiates the degradation of the second protein via a heat shock protein (Hsp). In certain embodiments, the first protein initiates the degradation of the second protein via heat shock protein 70 (Hsp70). In certain embodiments, the first protein is a hydrophobic tag (hyT).

In certain embodiments, the first protein is a ligase. In certain embodiments, the first protein is an ubiquitin ligase. In certain embodiments, the first protein is an E3 ligase. In certain embodiments, the E3 ligase is a HECT ligase, a RING-finger ligase, a U-box ligase, or a PHD-finger ligase. In certain embodiments, the ligase is SCFP-TrCP, von Hippel-Lindau (VHL), Murine double minute 2 (MDM2), an inhibitor of apoptosis protein (IAP), or cereblon (CRBN). In certain embodiments, the ligase is VHL.

In certain embodiments, the PROTAC is ARV-110, ARV-471, ARV-766, ARV-771, AVR-825, AR-LDD, DT2216, KT-474, KT-413, KT-333, NX-2127, NX-5948, CG001419, CFT8634, FHD-609, or SARD279. In certain embodiments, the PROTAC is ARV-771. In certain embodiments, the PROTAC is SARD279.

In another aspect, the present disclosure provides nanoparticles, comprising a conjugate disclosed herein, and a membrane encapsulating the conjugate.

In certain embodiments, the membrane is a lipid membrane. In certain embodiments, the membrane comprises 80-O14B. In certain embodiments, the membrane consists essentially of 80-014B.

In another aspect, the present disclosure provides pharmaceutical compositions, comprising a conjugate or nanoparticle disclosed herein, and a pharmaceutically acceptable excipient.

In another aspect, the present disclosure provides methods of treating diseases or disorders, comprising administering to a subject in need thereof a therapeutically effective amount of a conjugate or nanoparticle disclosed herein.

In certain embodiments, the disease or disorder is cancer. In certain embodiments, the cancer is selected from the group consisting of melanoma, brain cancer, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, and hepatocellular carcinoma. In certain embodiments, the cancer is a hematological cancer. In certain embodiments, the hematological cancer is myelogenous leukemia, myeloid leukemia, myelodysplastic syndrome, lymphoblastic leukemia, chronic lymphocytic leukemia (CLL), small lymphocytic lymphoma (SLL), high risk CLL, follicular lymphoma, diffuse large B-cell lymphoma (DLBCL), mantle cell lymphoma (MCL), Waldenstrom's macroglobulinemia (WM), multiple myeloma, marginal zone lymphoma (MZL), Burkitt's lymphoma, non-Burkitt high grade B cell lymphoma, extranodal marginal zone B cell lymphoma, transformed high grade B-cell lymphoma (HGBL), lymphoplasmacytic lymphoma (LPL), central nervous system lymphoma (CNSL), or MALT lymphoma. In certain embodiments, the cancer is a brain cancer. In certain embodiments, the cancer is neuroblastoma or glioblastoma.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the conjugate is preferably administered as a pharmaceutical composition comprising, for example, a conjugate of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a conjugate such as a conjugate of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a conjugate of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those conjugates, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The conjugate may also be formulated for inhalation. In certain embodiments, a conjugate may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the conjugate which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active conjugate, such as a conjugate of the invention, with the carrier and, optionally, one or more accessory ingredients. In general, the formulations are prepared by uniformly and intimately bringing into association a conjugate of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a conjugate of the present invention as an active ingredient. Compositions or conjugates may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium conjugates; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered conjugate moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active conjugates, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active conjugate may be mixed under sterile conditions with a pharmaceutically acceptable carrier, and with any preservatives, buffers, or propellants that may be required.

The ointments, pastes, creams and gels may contain, in addition to an active conjugate, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active conjugate, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a conjugate of the present invention to the body. Such dosage forms can be made by dissolving or dispersing the active conjugate in the proper medium. Absorption enhancers can also be used to increase the flux of the conjugate across the skin. The rate of such flux can be controlled by either providing a rate controlling membrane or dispersing the conjugate in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active conjugates in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject conjugates in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active conjugates can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a conjugate at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular conjugate or combination of conjugates employed, or the ester, salt or amide thereof, the route of administration, the time of administration, the rate of excretion of the particular conjugate(s) being employed, the duration of the treatment, other drugs, conjugates and/or materials used in combination with the particular conjugate(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or conjugate at levels lower than that required in order to achieve the desired therapeutic effect and gradually increase the dosage until the desired effect is achieved. By “therapeutically effective amount” is meant the concentration of a conjugate that is sufficient to elicit the desired therapeutic effect. It is generally understood that the effective amount of the conjugate will vary according to the weight, sex, age, and medical history of the subject. Other factors which influence the effective amount may include, but are not limited to, the severity of the patient's condition, the disorder being treated, the stability of the conjugate, and, if desired, another type of therapeutic agent being administered with the conjugate of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active conjugate used in the compositions and methods of the invention will be that amount of the conjugate that is the lowest dose effective to produce a therapeutic effect. Such an effective dose will generally depend upon the factors described above.

If desired, the effective daily dose of the active conjugate may be administered as one, two, three, four, five, six or more sub-doses administered separately at appropriate intervals throughout the day, optionally, in unit dosage forms. In certain embodiments of the present invention, the active conjugate may be administered two or three times daily. In preferred embodiments, the active conjugate will be administered once daily.

The patient receiving this treatment is any animal in need, including primates, in particular humans; and other mammals such as equines, cattle, swine, sheep, cats, and dogs; poultry; and pets in general.

In certain embodiments, conjugates of the invention may be used alone or conjointly administered with another type of therapeutic agent.

The present disclosure includes the use of pharmaceutically acceptable salts of conjugates of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit or promote degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

### EXAMPLES

The invention now being generally described, it will be more readily understood by reference to the following examples which are included merely for purposes of illustration of certain aspects and embodiments of the present invention, and are not intended to limit the invention.

### Example 1: Preparation of Exemplary Pre-Fused PROTACS

### Materials

ARV-771 was bought from MedChemExpress (Monmouth Junction, NJ, USA) and ARV-825 was purchased from LLCChemietek (Indianapolis, IN, USA). SARD279 was synthesized according to known literature methods. VHL and HSP70 proteins were both bought from Novus Biologicals (Centennial, CO, USA). 80-O14B lipid was synthesized according to known literature methods. HeLa and LNCaP cell lines were all purchased from ATCC (Manassas, VA, USA) and cultured in Eagle's minimal essential medium (DMEM) and Roswell Park Memorial Institute Medium (RPMI 1640) with 10% fetal bovine serum (FBS) and 100 μg/mL penicillin-streptomycin (Gibco Laboratories, Grand Island, NY, USA), respectively. Recombinant Anti-Brd4 (ab128874) and anti-AR (ab133273) antibodies were purchased from Abcam (Cambridge, MA, USA). Anti-GAPDH, HRP-labelled goat anti-rabbit, and HRP-labelled rabbit anti-mouse secondary antibodies were bought from Invitrogen (Carlsbad, CA, USA).

### Preparation and Encapsulation of Pre Fused PROATC

ARV-771 was firstly dissolved in DMSO and diluted to 100 nM in phosphate-buffered saline (PBS). Then VHL protein was added to the solution with a final concentration of 2.4 μg/mL. The mixture was incubated for 30 min at room temperature to form pre-fused ARV-771. 80-O14B LNP was formulated by directly dissolving the lipidoid into 25 mM sodium acetate solution (pH=5.5). 80-O14B LNP was added into the solution with the final concentration of 5 μg/mL and the solution was incubated at room temperature for another 15 min before administration. Pre-fused ARV-825 or SARD279 were prepared through the similar route.

### Example 2: Characterization of Exemplary Pre-Fused PROTACS

The encapsulation efficiency of ARV-771 and pre-fused ARV-771 were measured using high performance liquid chromatography (HPLC). ARV-771 and pre-fused ARV-771 LNPs were dialyzed (MWCO=3500) with water for 24 h. The LNPs were then freeze-dried and re-dissolved in acetonitrile. The concentration of ARV-771 encapsulated in the LNPs were measured by HPLC using the acetonitrile-water (90/10, V/V) as the mobile phase. The size and zeta potential of LNPs were characterized by Zetaplus analyzer (Zetaplus, Brookhaven, USA).

Generally, Western blots was carried out on the Invitrogen Novex SureLock Mini-Cell system. Supplies for the system were bought from Invitrogen (Carlsbad, CA, USA) 0.10 μL sample was loaded onto 4-12% bis-tris protein gel and the gel was run with a stable voltage of 120 V for 80 min. Then the gel was cut according to the protein ladder and transferred to PVDF membrane with a stable current of 250 mA for 3 h. The membrane was blocked by 5% skimmed milk for 1 h at room temperature and then incubated with primary antibody overnight. After 3 cycles of washing by TBST (tris-buffered saline, 0.1% tween 20), the membrane was incubated with secondary antibody for another 1 h and then washed by TBST for 3 times. The final membrane was then imaged using enhanced chemiluminescence (ECL) substrate.

### Example 3: Degradations of Exemplary Proteins by Exemplary Pre-Fused PROTACS

1×10⁶ HeLa cells were cultured in 6 well plate for 24 h before administration. Pre-fused ARV-771 was encapsulated into 80-O14B LNP and then diluted to different concentration. After 24 h incubation, the proteins were isolated from HeLa cells using the radio-immunoprecipitation assay (RIPA) lysis buffer and quantitated by bicinchoninic acid assay (BCA assay). Then the proteins were diluted to the sample concentration using NuPAGE LDS sample buffer (Invitrogen, Carlsbad, CA, USA) and denatured at 95° C. for 5 min. The study of targeted protein degradation by ARV-825 and SARD279 were carried out using similar protocols.

### Results and Discussion

### Enhanced Degradation of BRD4 by Delivery of Pre Fused ARV-771

ARV-771 was used as a model. There are two binding sites of ARV-771, one to bind an E3P known as von Hippel-Lindau (VHL) protein, and one to bind the POI, bromodomain-containing protein 4 (BRD4). BRD4, which promotes progression and regulation in different cancer types including neuroblastoma and glioblastoma, is considered an important therapeutic target. The PROTAC recruits and binds both VHL and BRD4, bringing the two compounds into close proximity. BRD4 is then ubiquitinated and subsequently degraded by proteasome. In traditional three-component PROTAC system, the ternary complex formed by binding of both BRD4 and VHL to AVR-771 is necessary for the successful degradation of BRD4. It was hypothesized that if VHL and ARV-711 are complex first before delivery, then the efficiency of BRD4 degradation would increase.

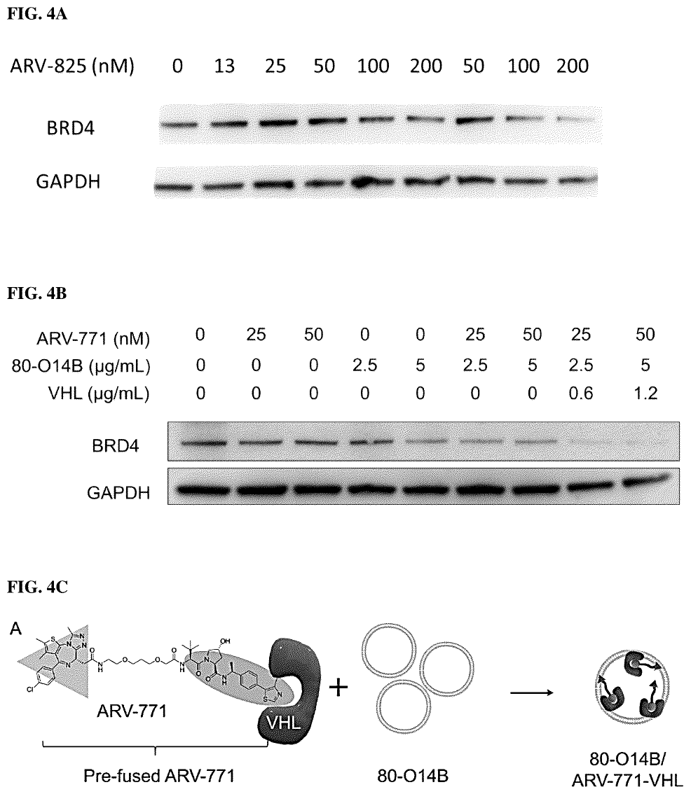

ARV-771 was firstly incubated with VHL protein in PBS for 30 min to from the pre-fused ARV-771 (FIG. 4C). Then the pre-fused ARV-771 was loaded into a synthetic lipidoid nanoparticle (LNP: 80-O14B). 80-O14B is a synthetic lipid nanoparticle that has previously been utilized for intracellular protein delivery. Following the encapsulation of pre-fused ARV-771, the LNP exhibited an increased particle size and reduced zeta potential, suggesting the successful interaction between LNP and pre-fused VHL/ARV-771 complex. The encapsulation efficiency of unfused ARV-771 in 80-O14B LNP is approximately 27.2%, whereas the encapsulation efficiency is approximately 60.8% using pre-fused ARV-771. The increase in the ARV-771 encapsulation may be attributed to the high binding rate of VHL-protein to cationic LNPs.

HeLa cells, which express BRD4 at high levels, were selected as a model cell line to evaluate the efficiency of pre-fused ARV-771-induced BRD4 degradation (FIG. 4A). After 24 h of treatment with unfused ARV-771 at concentrations of 25 and 50 nM, the BRD4 levels remained at high (e.g., more than 80% remained). The control cells treated with blank LNP didn't show significant degradation of BRD4, indicating that the lipid nanoparticles themselves have no proteolytic activity. Notably, unfused ARV-771-loaded LNP showed an enhancement of BRD4 degradation, due to the improved delivery of large PROTAC molecule (ARV-771) by LNP. However, after fusing ARV-771 with VHL and subsequently loading the fused molecule into LNPs for intracellular delivery, BRD4 were almost completely degraded (e.g., >90% reduction) after 24 hours (FIG. 4B). Thus, the perfused encapsulated PROTACS outperformed the unfused encapsulated PROTACs by a significant margin.

The degradation of BRD4 at different concentration of VHL was determined by Western blot. The VHL pre-fused ARV-711 exhibited increased degradation of BRD4 when the concentration of ARV-711 increased in the range of 0.16-0.63 μg/mL. However, the continuous increasing of VHL concentration to 5 μg/mL didn't further increase the BRD4 degradation, possibly due to the saturation of the binding of VHL and ARV-771. Nonetheless, the effect of pre-fusing VHL to ARV-711 on the degradation of BRD4 further demonstrated the important role of VHL in the pre-fusion system.

To investigate whether the enhanced protein degradation was truly induced by the pre-fusion of PROTAC with its corresponding E3P, investigations with another PROTAC (ARV-825), were performed. ARV-825 is also a BRD4 degrader, however its UPS-activating ligand binds to cereblon instead of VHL. As ARV-825 lacks a VHL binding site, the incubation of ARV-825 with VHL should not form stable pre-fused PROTAC. The pre-incubation of ARV-825 with VHL didn't show enhanced BRD4 degradation compared with that of free ARV-825, even at the high concentration of 100 nM. Furthermore, the delivery of VHL protein alone also showed no effect on protein degradation. These results demonstrated the enhanced degradation of POI (e.g., BRD4) can be attributed to the delivery of pre-fused PROTACs.

### Increased Level and Rate of Protein Degradation by Delivery of Pre Fused PROTAC

The degradation kinetics of free ARV-771 and pre-fused ARV-771 were further evaluated. Free AVR-771 showed modest degradation of BRD4 with the half degradation concentration (DC₅₀) of about 100 nM (FIG. 4B). However, after pre-fusion with VHL and delivery by 80-O14B LNP, the degradation ability of BRD4 increased significantly (FIG. 4B). BRD4 was almost complete degraded (e.g., >95%) at a concentration of 25 nM.

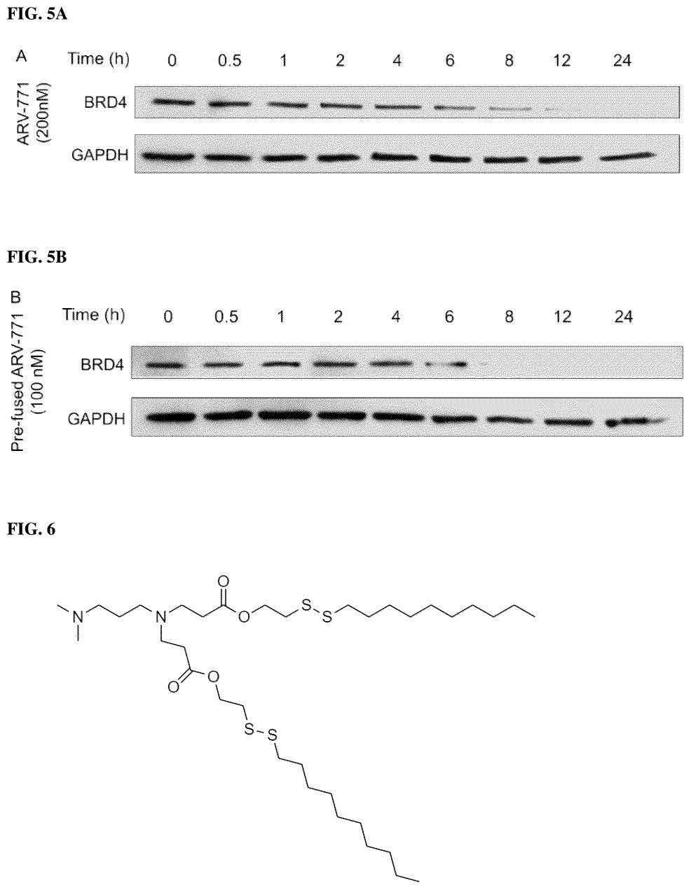

The two-component PROTAC system not only increases the total amount degradation, but also enhances the rate of the degradation because of the simplified degradation route, as compared to that of three-component system (FIG. 1B). For the three-component PROTAC system, there are at least two steps before the formation of the ternary complex required for ubiquitination and degradation of POIs. Specifically, both the POIs and E3Ps must bind to the same PROTAC molecule, which increases the time for successful conjugate degradation. However, in the two-component system, after the pre-fusion with E3Ps, only the binding of the targeted proteins is required for the formation of ternary complex before the successful ubiquitination and degradation. Such a simplified binding route may accelerate the speed of degradation. To test this hypothesis, a time-dependent degradation assay in was performed (FIG. 5A). HeLa cells were treated with free ARV-771 or VHL pre-fused ARV-771 for different time periods. For free ARV-771, no significant degradation of BRD4 was observed during the first 6 h. Small amounts of degradation were observed after 8 h and the total degradation occurred only after 24 h of incubation (FIG. 5A). However, the degradation rate was significantly accelerated in the cells treated with VHL pre-fused ARV-771 delivered using 80-O14B LNP. BRD levels were reduced by approximately 50% after only 6 hours and degradation was almost complete (e.g., >90%) by the 8 h point (FIG. 5B).

Simply put pre-fusion of PROTAC not only increased the degradation level of BRD4 protein, but also accelerated the degradation rate.

### Pre-Fusion as a General Method for Enhancing the Efficiency of Other Three-Component Systems

To further investigate whether the pre-fusion method can be utilized as a general strategy for enhancing protein degradation, degradation SARD279 using was studied. SARD279 is a HyT (hydrophobic tag) degrader for the androgen receptor (AR). The UPS-activating ligands of HyT degraders are hydrophobic molecules that can recruit HSP70 for targeted protein degradation.

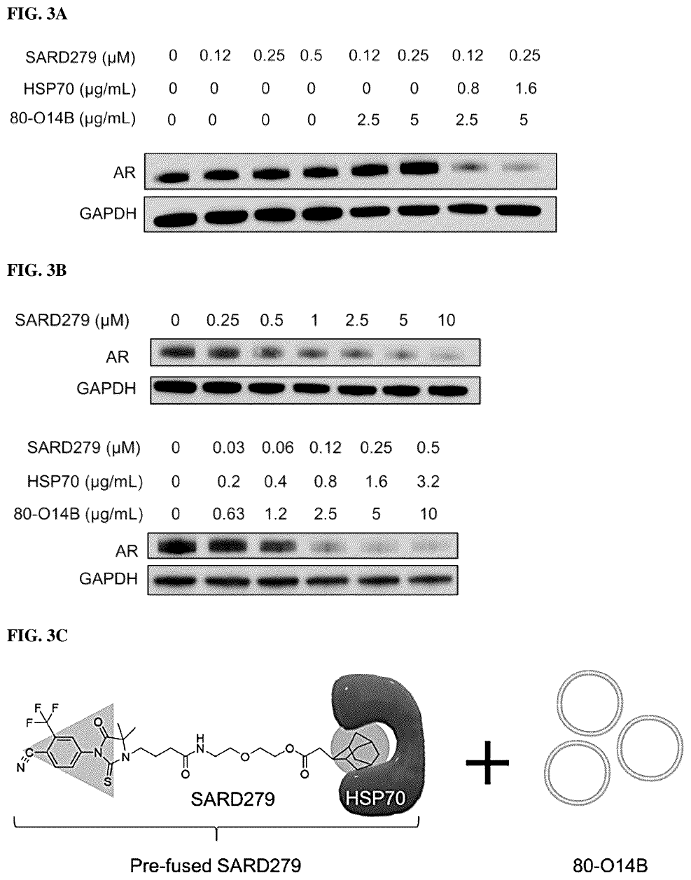

Free SARD279 showed no AR degradation in LNCaP cells at the concentration from 0.125 to 0.5 μM (FIG. 3B). The addition of LNP to the system did not increase the AR degradation efficiency. However, after pre-fusing SARD279 with HSP70 and delivering using LNP, the complex showed more than 80% degradation of AR at 0.5 μM (FIG. 3C). Moreover, the degradation of AR by pre-fused SARD279 showed a concentration de-pendency. The DC₅₀ of SARD279 after pre-fusion decreased to about 0.06 μM, about 16 times lower than that of the free SARD279 (FIG. 3B).

The enhancement of SARD279 by the pre-fusion of HSP70 further demonstrated the pre-fusion of E3P to PROTACs could be a universal method to increase efficiency of PROTAC mediated protein degradation.

### SUMMARY

Disclosed herein is a novel two-component PROTAC system by the pre-fusing E3Ps to PROTACs before administration and delivered it to the targeted cells by LNPs for enhanced degradation of POIs. As a proof of concept, VHL to ARV-771 was pre-fused and delivered it to HeLa cells for degradation of BRD4. The two-component PROTAC system significantly increased the efficiency of the degradation of POI as compared to a three-component system, even at low concentrations. Moreover, the rate of degradation was also accelerated in the two-component system, as compared to the three-component system. Finally, the efficacy of this strategy was also confirmed in Hyts degraders, revealing the versatility of this method.

### INCORPORATION BY REFERENCE

All U.S. patents and published U.S. and PCT applications mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A conjugate, comprising a proteolysis-targeting chimera (PROTAC), and a first protein; wherein the PROTAC and the first protein are covalently or non-covalently bonded to each other; and the first protein initiates degradation of a second protein.

2. The conjugate of claim 1, wherein the PROTAC and the first protein are covalently bonded to each other.

3. The conjugate of claim 1, wherein the PROTAC and the first protein are non-covalently bonded to each other.

4. The conjugate of claim 1, wherein the first protein initiates the degradation of the second protein via the ubiquitin-proteasome system (UPS).

5. The conjugate of claim 1, wherein the first protein initiates the degradation of the second protein via a heat shock protein (Hsp).

6. The conjugate of claim 5, wherein the first protein initiates the degradation of the second protein via heat shock protein 70 (Hsp70).

7. The conjugate of claim 1, wherein the first protein is a ligase.

8. The conjugate of claim 1, wherein the first protein is an ubiquitin ligase.

9. The conjugate of claim 1, wherein the first protein is an E3 ligase.

10. The conjugate of claim 9, wherein the E3 ligase is a HECT ligase, a RING-finger ligase, a U-box ligase, or a PHD-finger ligase.

11. The conjugate of claim 1, wherein the ligase is SCFβ-TrCP, von Hippel-Lindau (VHL), Murine double minute 2 (MDM2), an inhibitor of apoptosis protein (IAP), or cereblon (CRBN).

12. The conjugate of claim 11, wherein the ligase is VHL.

13. The conjugate of claim 1, wherein the first protein is a hydrophobic tag (hyT).

14. The conjugate of claim 1, wherein the second protein is androgen receptor, an estrogen receptor, bromodomain (BRD) protein, Bromo- and Extra-Terminal domain (BET) protein, B-cell lymphoma-extra large (Bcl-xL) protein, interleukin receptor (IL-R), Interleukin-1 receptor associated kinase (IRAK), signal transducer and activator of transcription protein (STAT) (e.g., STAT 3), Bruton's tyrosine kinase (BTK), tyrosine receptor kinase (TRK),

15. The conjugate of claim 1, wherein the PROTAC is ARV-110, ARV-471, ARV-766, ARV-771, AVR-825, AR-LDD, DT2216, KT-474, KT-413, KT-333, NX-2127, NX-5948, CG001419, CFT8634, FHD-609, or SARD279.

16. The conjugate of claim 1, wherein the PROTAC is ARV-771.

17. The conjugate of claim 1, wherein the PROTAC is SARD279.

18. A nanoparticle, comprising the conjugate of claim 1; and a membrane encapsulating the conjugate.

19.-21. (canceled)

22. A pharmaceutical composition, comprising the conjugate of claim 1 and a pharmaceutically acceptable excipient.

23. A method of treating a disease or disorder, comprising administering to a subject in need thereof a therapeutically effective amount of the conjugate of claim 1.

24.-29. (canceled)
